(19) United States
(12) Patent Application Publication
(10) Pub. No.: US 2011/0159113 A1
(43) Pub. Date: Jun. 30, 2011

(54) HYPERBRANCHED POLYESTER AND A METHOD OF SYNTHESIZING A HYPERBRANCHED POLYESTER

(76) Inventors: MOHSEN ADELI, Khoramabad (IR); Farzaneh Saadatmehr, Khoramabad (IR); Mahdieh Kalantari, Khoramabad (IR); Bahram Rasoolian, Khoramabad (IR)

(21) Appl. No.: 13/044,053

(22) Filed: Mar. 9, 2011

(51) Int. Cl. A61K 33/24; C07C 69/675

(52) U.S. Cl. 424/649; 560/182

(57) ABSTRACT

The various embodiments herein provide a hyper branched polyester and a method of synthesizing the same. The embodiments herein also provide a method of encapsulating a drug into the void spaces of the polymer to act as a drug delivery system. The hyper branched polyester mer comprises an acidic moiety and an alcoholic moiety. The acidic moiety is citric acid monohydrate. The alcoholic moiety is glycerol. The acidic moiety and the alcoholic moiety are randomly arranged in the hyperbranched polymer. The method comprises of heating the mixture of citric acid and glycerol at temperatures of 90 to 150° C. by constant stirring for different time periods. In the method of encapsulation, the drug solution is drop-wise added to polymer solution at 37° C. by constant stirring for 24 h.

### SPONSORSHIP STATEMENT

The present invention for international filing is sponsored by The Iranian Nanotechnology initiative Council.

### BACKGROUND

1. Technical Field

The embodiments herein generally relate to the field of polymer production and particularly to a dendritic polymers. The embodiments herein more particularly relate to a hyper branched polymer and a method of synthesis of hyper branched polyesters using an acidic moiety and alcoholic moiety. The embodiments herein also relate to a method of encapsulation of drug for use as a drug delivery system.

2. Description of the Related Art

A wonderful and well-known case study in the growth of science is found in the birth of polymer chemistry in the early 20th century. Now, at the beginning of the 21st century, polymers can be classified into four groups based on their properties and architecture such as (a) linear, random coil thermoplastics, (b) cross-linked thermosets, (c) branched systems based on long-chain branching in polyolefins, and (d) dendritic polymers.

Dendritic polymers consist of three subgroups namely random hyper branched polymers, dendrigraft polymers and dendrimers. Dendritic polymers have excellent chemical and physical properties compared to other types of polymers. Dendritic polymers are a relatively young class of polymers but well established body of interdisciplinary research exploring a remarkable variety of potential applications. Dendrimers and hyper branched polymers are characterized by highly branched structures, large numbers of functional end groups, low intrinsic viscosities and very high solubilities. The low intrinsic viscosity is attributed to their packed structure, while the high solubility is a result of the large number of functional end groups available per macromolecule. Dendrimers have a wide range of applications such as drug transport, gene transport systems, high-loading supports for organic synthesis, water purification systems, and molecular nanocarriers. However large-scale use of dendrimers is restricted because of their labor-intensive synthesis and the resulting limited availability in bulk quantities.

Unlike dendrimers, the hyperbranched polymers (HBs), are relatively inexpensive to produce and are easy to synthesize in large quantities through simple methods that do not need the tedious isolation and purification procedures. Therefore, hyperbranched polymers as a unique type of dendritic polymers can be an attractive alternative to dendrimers because they possess both interesting properties of the dendritic structures and also feasibility for large-scale manufactures.

Over the past 10 years, hyperbranched polymers (HBs) have been suggested for a broad range of applications. Most of the applications of hyperbranched polymers are based on their good solubility and the large number of functional groups within a molecule. Hyperbranched polyesters are an important class of hyperbranched polymers, and the availability of inexpensive raw materials has prompted many research groups to investigate hyperbranched polyesters in details.

Hence there is a need for a cheaper, faster and easy method of synthesis of hyper branched polymer that is used in a wide variety of applications.

The above mentioned shortcomings, disadvantages and problems are addressed herein and which will be understood by reading and studying the following specification.

### OBJECTIVES OF THE EMBODIMENTS

The primary object of the embodiments herein is to provide a new method for the synthesis of a hyper branched polymer using an acidic moiety and alcoholic moiety.

Another object of the embodiments herein is to provide a method of synthesis of a hyper branched polyester based on acidic moiety and alcoholic moiety, in which acidic and alcoholic moieties comprise at least three acidic and three hydroxyl functional groups respectively.

Yet another object of the embodiments herein is to provide a method of synthesis of hyperbranched polyesters based on citric acid as an AB3 monomer and glycerol as an A3 monomer.

Yet another object of the embodiments herein is to provide a method of synthesis of a hyper branched polyester at different citric acid/glycerol molar ratios.

These and other objects and advantages of the embodiments herein will become readily apparent from the following detailed description taken in conjunction with the accompanying drawings.

### SUMMARY OF THE EMBODIMENTS

The various embodiments herein provide a hyper branched polyester and a method to synthesis the hyperbranched polyester. The hyperbranched polyester is synthesized using monomers with acidic and alcoholic functional groups in which acidic and alcoholic monomers contains at least three acidic and hydroxyl functional groups respectively. Synthesized hyperbranched polyesters are promising candidates and used in a wide variety of applications and particularly in biomedical applications. The hyperbranched polyester is prepared from acidic and alcoholic monomers using different methods. The acidic moiety comprises at least three acidic functional groups. The acidic moiety comprises at least one type of acidic compound. The acidic moiety comprises different types of acidic compounds. The alcoholic moiety comprises at least three hydroxyl functional groups. The alcoholic moiety comprises one type of alcoholic compound. The alcoholic moiety comprises different types of alcoholic compounds. The hyperbranched polyester is promising candidate in order to use in variety of applications particularly biomedical applications.

According to an embodiment herein, the hyperbranched polyester based on citric acid as an AB₃ monomer and glycerol as an A₃ monomer with different molar ratios of citric acid/glycerol are synthesized.

According to an embodiment herein, the hyper branched polyesters comprise an acidic moiety and an alcoholic moiety. The acidic moiety is citric acid monohydrate. The alcoholic moiety is glycerol. The acidic moiety and the alcoholic moiety are randomly arranged in the hyper branched polyester.

The acidic moiety comprises at least three acidic functional groups. The acidic moiety comprises one or more types of acidic compounds. The acidic moiety comprises one type of acidic compound. The acidic moiety is citric acid monohydrate.

The alcoholic moiety comprises at least three hydroxyl functional groups. The alcoholic moiety comprises one or more types of alcoholic compounds. The alcoholic moiety comprises one type of alcoholic compound. The alcoholic moiety is glycerol.

A method of synthesizing a hyper branched polyester comprises mixing a predetermined amount of citric acid monohydrate (CA) and a predetermined amount of glycerol (G) in a polymerization ampule to prepare a first solution. The citric acid and glycerol are mixed at a temperature of 90° C. The prepared first solution is heated at a temperature of 110° C. for 20 min by constantly stirring the first solution. The temperature of the first solution in the polymerization ampule is further increased to 120° C. and the first solution is stirred for next 30 min simultaneously. The first solution is stirred at a plurality of temperature levels for a plurality of time intervals successively. The first solution is stirred at 130° C. for 40 min. The first solution is stirred at 140° C. for 40 min. The first solution is stirred at 145° C. for 50 min and the first solution is stirred at 150° C. for 60 min. The first solution is heated and stirred successively at the plurality of temperature levels under vacuum to remove a water. The first solution is kept at a room temperature to cool down to form a viscose compound.

The viscose compound is dissolved in tetrahydrofuran (THF) to form a second solution. The second solution is a viscous solution. The second solution is filtered to obtain a third solution. The third solution is a clear solution. The third solution is concentrated under a reduced pressure to obtain a second compound. The second compound is precipitated in a cyclohexane solvent. The precipitated second compound is dialysed against a THF solvent to obtain a fourth solution. The fourth solution is evaporated under a reduced pressure to obtain a pure product of hyper branched polyester. The hyper branched polyester is obtained in different molar ratios.

The citric acid monohydrate and glycerol are mixed in a plurality of molar ratios of citric acid monohydrate/glycerol (CA/G). The plurality of molar ratios of citric acid monohydrate/glycerol (CA/G) includes 5, 8 and 12. The citric acid monohydrate and glycerol are mixed in a molar ratio of 5.

The predetermined amount of citric acid monohydrate added to glycerol is 7 g (33 mmol) or 11.09 g (52.8 mmol) or 16.64 g (79.2 mmol). The predetermined amount of glycerol added to citric acid monohydrate is 0.5 ml (6.6 mmol).

The precipitated compound is dialyzed against THF solvent for 4 h, 6 h and 8 h. The precipitated compound is against THF solvent dialyzed for 8 h. The method of synthesizing the hyper polyester is a melt polycondensation method.

A method of encapsulating a drug into a hyper branched polyester comprises dissolving the hyper branched polyester in distilled water. The amount of hyper branched polyester dissolved in 1 ml of distilled water is 0.1 gm (1.67×10⁻² mmol). A drug solution with a preset quantity at a preset concentration is prepared. The preset quantity of the drug solution is 100 ml and the preset concentration of the drug solution is 50 μg/ml. The drug solution is added to the dissolved hyper branched polyester solution to obtain a mixture solution. The drug solution is added the dissolved hyper branched polyester solution in drop-wise. The mixture solution is stirred at 37° C. for 24 h in a dark condition to obtain an encapsulated drug. The drug molecules are encapsulated into a void space of the hyper branched polyester in the encapsulated drug.

The hyper branched polyester is obtained by mixing a preset quantity of citric acid and a preset quantity of glycerol in a molar ratio of 5, 8 and 12. The preset quantity of citric acid is 7 g (33 mmol) or 11.09 g (52.8 mmol) or 16.64 g (79.2 mmol) and the preset quantity of glycerol is 0.5 ml (6.6 mmol). The drug is cisplatin. The hyper branched polyester encapsulated by a drug is used for drug delivery.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining a method of synthesizing the hyper branched polymer or polyester, according to one embodiment herein.

FIG. 2 shows a preparation route of hyper branched polymer using citric acid (CA) and glycerol (G) monomers forming Poly (citric acid-co-glycerol) or P (CA-G) polymer, according to an embodiment herein.

FIG. 3A illustrates a representation of poly (citric acid-co-glycerol) or P (CA-G) polymer, according to one embodiment herein.

FIG. 3B illustrates Poly (citric acid-co-glycerol)-Cis-Diamminedichloroplatinum complex or a P(CA-G)-CDDP complex, according to an embodiment herein.

FIG. 4 shows an Infra red (IR) spectrum of the synthesized hyperbranched polymer with a citric acid/glycerol (CA/G) molar ratio of 5, P(CA5-G), according to an embodiment herein.

FIG. 5 shows a Hydrogen Nuclear Magnetic Resonance (HNMR) spectrum of the synthesized hyperbranched polymer with a citric acid/glycerol (CA/G) molar ratio of 5, P(CA₅-G), according to an embodiment herein.

FIG. 6 shows a HNMR spectrum of the synthesized hyperbranched polymer with a citric acid/glycerol (CA/G) molar ratio of 8, P(CA₈-G), according to an embodiment herein.

FIG. 7 shows a Carbon Nuclear Magnetic Resonance (CNMR) spectrum of the synthesized hyperbranched polymer with a citric acid/glycerol (CA/G) molar ratio of 5, P(CA₅-G), according to an embodiment herein.

FIG. 8 shows a CNMR spectrum of the synthesized hyperbranched polymer with a citric acid/glycerol (CA/G) molar ratio of 8, P(CA₈-G), according to an embodiment herein.

FIG. 9 shows a Gel permeation chromatography (GPC) diagrams of synthesized hyperbranched polymer with different molar ratios, where (a) shows the GPC diagram for the synthesized hyperbranched polymer with a CA/G molar ratio of 12, P(CA₁₂-G), where (b) shows the GPC diagram for the synthesized hyperbranched polymer with a CA/G molar ratio of 8, P(CA₈-G) and where (c) shows the GPC diagram for the synthesized hyperbranched polymer with a CA/G molar ratio of 5, P(CA₅-G), according to an embodiment herein.

FIG. 10 shows a Dynamic light scattering (DLS) diagrams of the synthesized hyperbranched polymer with molar ratios of 8 and 12, where (a) shows the DLS diagram of the synthesized hyperbranched polymer with molar ratios of 8, P(CA₈-G) and where (b) shows the DLS diagram of the synthesized hyperbranched polymer with molar ratios of 12, P(CA₁₂-G), according to an embodiment herein.

FIG. 11 shows a graphical representation of the Zeta potential of synthesized hyperbranched polymer with molar ratios of 8 and 12, where (a) shows the zeta potential value for the synthesized hyperbranched polymer with a molar ratio of 8, P(CA₈-G) and where (b) shows the zeta potential value for the synthesized hyperbranched polymer with a molar ratio of 12, P(CA₁₂-G), according to an embodiment herein.

FIG. 12 shows a Thermo-gravimetric analyses (TGA) thermo grams of the synthesized hyperbranched polymers, where (a) shows a TGA thermogram for polymer with a molar ratio of 5, P(CA5-G), synthesized with a total reaction time of 4 h, where (b) shows a TGA thermogram for polymer with a molar ratio of 8, P(CA8-G), synthesized with total reaction time of 4 h and where (c) shows a TGA thermogram for polymer with a molar ratio of 5, P(CA5-G), synthesized with total reaction time of 6 h, according to an embodiment herein.

FIG. 13 shows a MTT (3-(4,5-dimethylthiazol-2-yl)-2,5-diphenyltetrazolium bromide) assay results for Cis-Diamminedichloroplatinum CDDP, P(CA₁₂-G)-CDDP and P(CA₈-G)-CDDP, according to an embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The embodiments herein relates to the synthesis of hyperbranched polyesters from acidic and alcoholic monomers. These acidic and alcoholic monomers have at least three acidic and hydroxyl functional groups, respectively. According to an embodiment herein, hyperbranched polyesters based on citric acid (CA) as AB3 monomer and glycerol (G) as A3 monomer are synthesized. The building blocks of the synthesized hyperbranched polymer are only citric acid and glycerol which are attached together randomly. Citric acid is a cheap and biocompatible compound that is used on a large scale in the food and drug industries. On the other hand glycerol is a key component in the synthesis of phospholipids. Therefore, the polymers based on citric acid and glycerol possesses unique properties.

Increasing the molar ratio of citric acid/glycerol means increasing the citric acid building blocks in the obtained copolymer. With increased citric acid/glycerol molar ratio, the number of the carboxyl functional groups of copolymer increases, the negative surface charge of copolymer rises, the size of copolymer increase and the transport capacity of copolymers to transfer small molecules with positive charge increases.

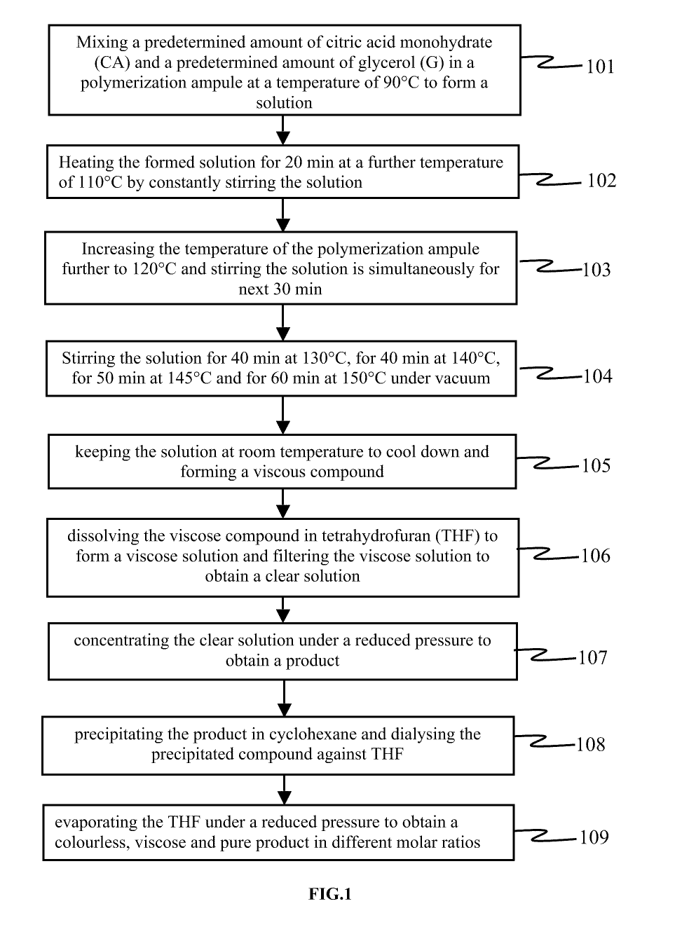

FIG. 1 illustrates a block diagram showing the steps of synthesis of the hyperbranched polymer or polyester, according to one embodiment herein. With respect to FIG. 1, a predetermined amount of citric acid monohydrate (CA) and a predetermined amount of glycerol (G) are mixed in a polymerization ampule at a temperature of 90° C. to form a solution (101). The formed solution is then heated for 20 min at a temperature of 110° C. by constantly stirring the solution (102). The temperature of the polymerization ampule is increased further to 120° C. and the solution is simultaneously stirred for next 30 min at this temperature (103). The solution is further stirred for 40 min at 130° C., for 40 min at 140° C., for 50 min at 145° C. and for 60 min at 150° C. under vacuum (104). The solution is kept at room temperature to cool down to form a viscous compound (105). The viscose compound is dissolved in tetrahydrofuran (THF) solvent to form a viscous solution and the viscous solution is filtered to obtain a clear solution (106). The clear solution is concentrated under a reduced pressure to obtain a product (107). The product is precipitated in cyclohexane and dialyzed against THF (108). The dialyzed product in THF is evaporated under a reduced pressure to obtain a pure, colourless and viscose product in different molar ratios (109). The citric acid monohydrate and glycerol are mixed in different citric acid monohydrate/glycerol (CA/G) molar ratios of 5, 8 and 12. The molar ratio is 5. The predetermined amount of citric acid monohydrate is 7 g (33 mmol), 11.09 g (52.8 mmol) and 16.64 g (79.2 mmol) and the predetermined amount of glycerol is 0.5 ml (6.6 mmol) to form molar ratios (CA/G) of 5, 8 and 12, respectively. The precipitated compound is dialyzed against THF for 4 h, 6 h and 8 h. The precipitated compound is dialyzed for 8 h. The method of synthesizing the poly (citric acid-co-glycerol) is a melt polycondensation method.

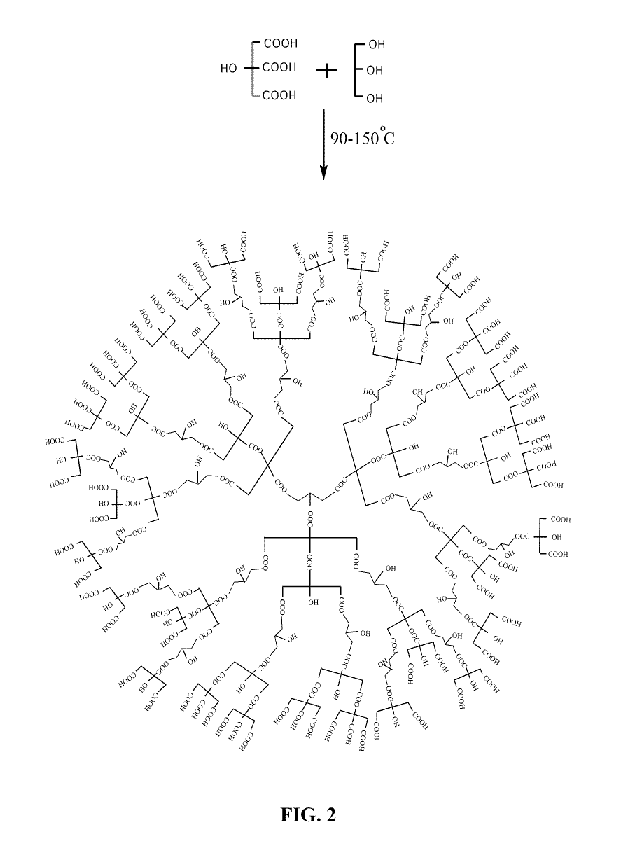

FIG. 2 illustrates the preparation route of hyperbranched polymer using citric acid (CA) and glycerol (G) monomers forming Poly (citric acid-co-glycerol) or P (CA-G) polymer, according to an embodiment herein. With respect to FIG. 2, the citric acid and glycerol are mixed and heated at a temperature between 90 to 150° C. This is a melt polycondensation method. The melt polycondensation is a synthetic route of forming the hyperbranched polymers. According to an embodiment herein, condensation polymerization of the AB3 monomer citric acid in the presence of the glycerol as B3 monomer with different CA/G molar ratios leads to the formation of hyperbranched poly citric acid-glycerol.

Although the development of pharmaceutical biotechnologies have led to an increasing number of new drugs, the drugs still possess many intrinsic limitations to large-scale applications, such as low biocompatibility and poor solubility. Since all of the intrinsic properties of a drug are fixed after synthesis, the design of an appropriate delivery system can be used as a promising way to overcome such problems. So, the hyper branched polymers are used as a drug delivery system with an increased efficiency and solubility. The hyper branched polymers possess cavities in which drug molecules are encapsulated. According to an embodiment herein, cisplatin, an anticancer drug, is encapsulated into the void spaces of hyper branched polyester. Cis-platinum complexes are widely used for treatment of a wide spectrum of cancers such as lung, ovarian, head and neck cancer. Cisplatin performs its antitumor activity by forming stable DNA-cisplatin complexes through intra-strand crosslinks. This results in interference with normal transcription and DNA replication mechanisms leading to apoptosis. However, toxicity and poor water solubility of cisplatin limit its high cancer activity.

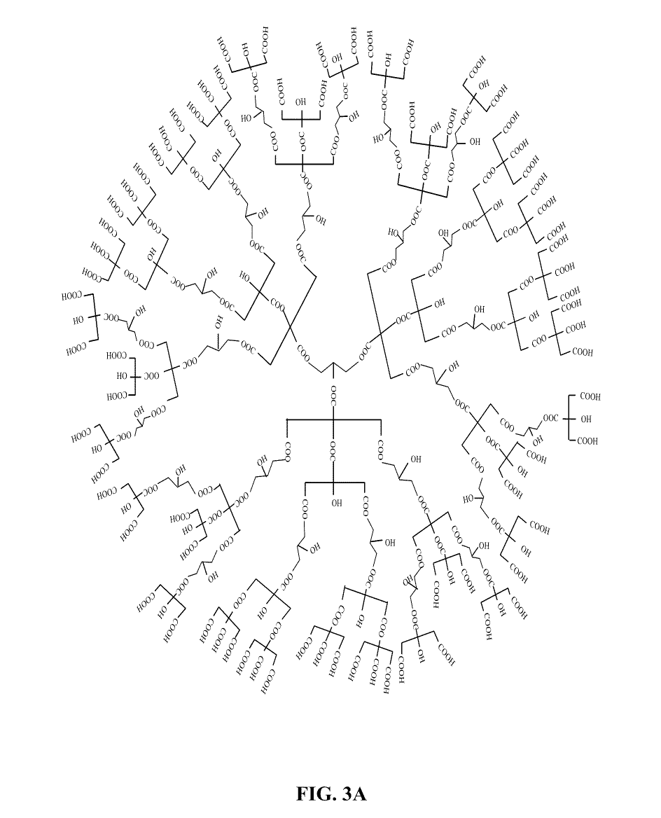

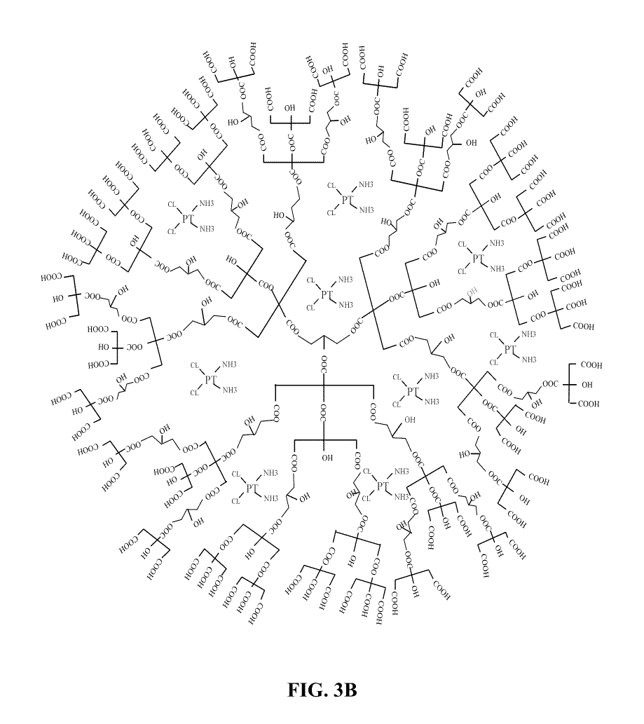

FIG. 3A illustrates a representation of poly (citric acid-co-glycerol) or P (CA-G) polymer, according to one embodiment herein. With respect to FIG. 3A, the citric acid molecules are cross linked randomly to form the poly (citric acid-co-glycerol) hyperbranched polymer.

FIG. 3B illustrates Poly (citric acid-co-glycerol)-Cis-Diamminedichloroplatinum complex or a P(CA-G)-CDDP complex, according to an embodiment herein. With respect to FIG. 3B, the encapsulation of Cis-Diamminedichloroplatinum (CDDP) into void spaces of Poly (citric acid-co-glycerol) can be observed. The P(CA-G)-CDDP complex is used as drug delivery system for delivery of cis-platin drug inside a body.

With respect to FIG. 3A and FIG. 3B, the cisplatin solution is added to the huperbranched polymer solution drop wise under constant stirring for 24 h at 37° C. so as to form an encapsulated P(CA-G)-CDDP complex. There are two ways to transport anticancer drugs by copolymers. They are encapsulation and complexation routes. Complexation route is assigned to the absorption of anticancer drugs onto the surface of copolymers by electrostatic interaction or coordination of metal atoms of drugs such as CDDP to the carboxyl functional groups of copolymers. The Encapsulation is an entrapment of the drug molecules into the void spaces to polymer or copolymer or hyperbranched polymer.

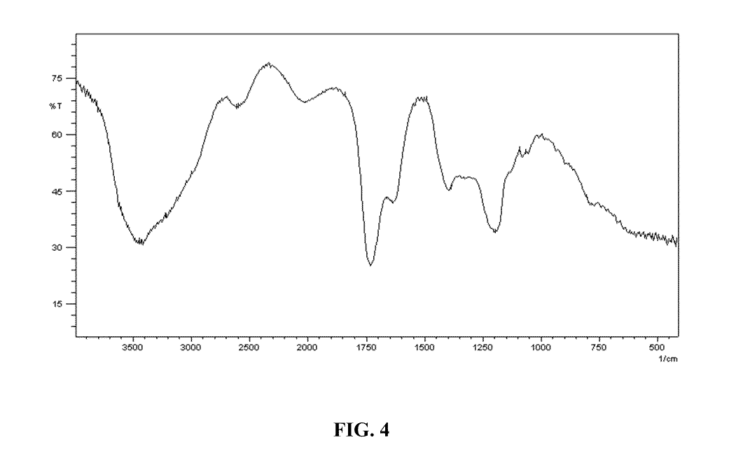

FIG. 4 shows an Infra red (IR) spectrum of the synthesized hyperbranched polymer with a citric acid/glycerol (CA/G) molar ratio of 5, P(CA5-G), according to an embodiment herein. With respect to FIG. 4, carbonyl bands at 1647 and 1731 cm⁻¹ are assigned to the acid and ester groups of hyperbranched polyester, respectively. The presence of ester vibration in the IR spectrum is a result of the polymerization of citric acid.

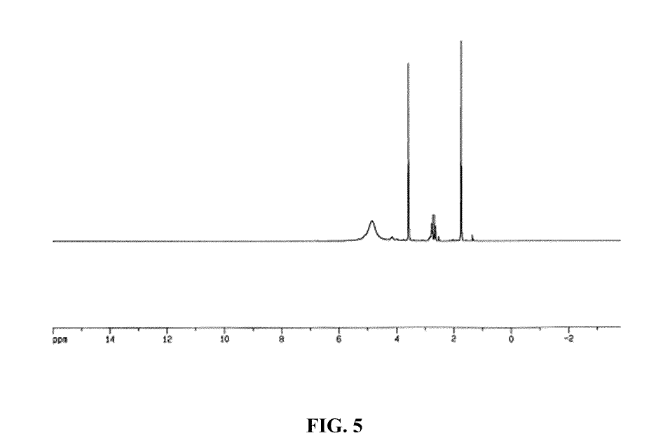

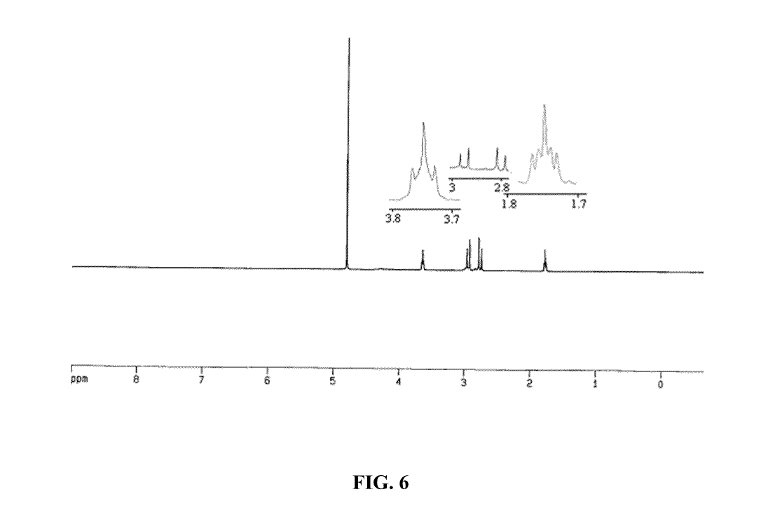

FIG. 5 shows a Hydrogen Nuclear Magnetic Resonance (HNMR) spectrum of the synthesized hyperbranched polymer with a citric acid/glycerol (CA/G) molar ratio of 5, P(CA₅-G), according to an embodiment herein. FIG. 6 shows a HNMR spectrum of the synthesized hyperbranched polymer with a citric acid/glycerol (CA/G) molar ratio of 8, P(CA₈-G), according to an embodiment herein. With respect to FIG. 5 and FIG. 6, the peaks attributed to the citric acid and glycerol are found. FIG. 5 and FIG. 6 show peaks at 2.5-2.7 ppm due to the protons of citric acid. Signals of glycerol are appeared at 1.7 and 3.6 ppm for CH and CH2 groups, respectively. In addition, FIG. 5 reveals the Signal at about 4.85 ppm for water molecules in the polymer matrix. The comparison of the integrated peak areas for protons in P(CA₅-G) and P(CA₈-G) spectra indicates that the average number of reacted citric acid monomers increases with increasing CA/G molar ratio (integration values not shown in the ¹H-NMR spectra).

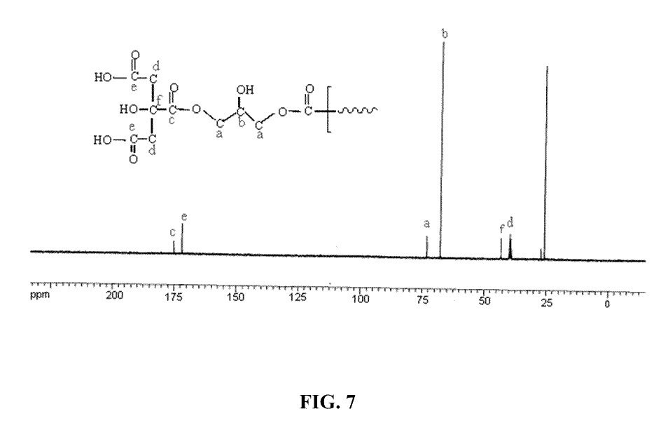

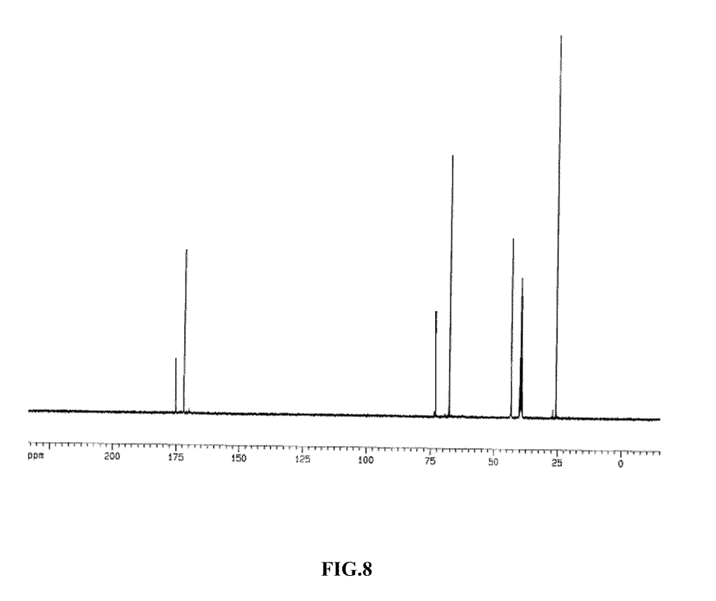

FIG. 7 shows a Carbon Nuclear Magnetic Resonance (CNMR) spectrum of the synthesized hyperbranched polymer with a citric acid/glycerol (CA/G) molar ratio of 5, P(CA₅-G), according to an embodiment herein. FIG. 8 shows a CNMR spectrum of the synthesized hyperbranched polymer with a citric acid/glycerol (CA/G) molar ratio of 8, P(CA₈-G), according to an embodiment herein. With respect to FIG. 7 and FIG. 8, all the peaks attributed to glycerol (a, b), citric acid (f, d), acid (e) and ester (c) groups are observed. According to the integration of peaks in P(CA₅-G) and P(CA₈-G) spectra (integration values not shown in the CNMR spectra), hyperbranched polyester synthesized with a CA/G molar ratio of 8 has higher citric acid units which is in good agreement with H-NMR results. NMR spectra of P(CA₅-G) and P(CA₈-G) were obtained in DMSO-d₆ and D₂O, respectively.

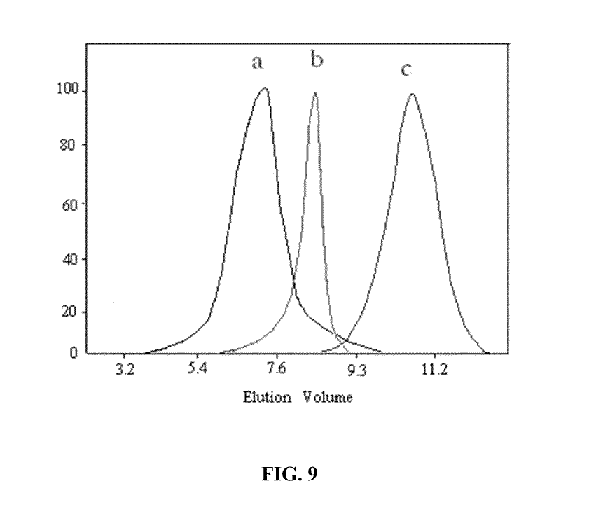

In order to measure the molecular weight of hyperbranched polyester and study the effect of CA/G molar ratio on it, GPC experiments were performed using polymers synthesized with different CA/G molar ratios. FIG. 9 shows a Gel permeation chromatography (GPC) diagrams of synthesized hyperbranched polymer with different molar ratios, where (a) shows the GPC diagram for the synthesized hyperbranched polymer with a CA/G molar ratio of 12, P(CA₁₂-G), where (b) shows the GPC diagram for the synthesized hyperbranched polymer with a CA/G molar ratio of 8, P(CA₈-G) and where (c) shows the GPC diagram for the synthesized hyperbranched polymer with a CA/G molar ratio of 5, P(CA₅-G), according to an embodiment herein. With respect to FIG. 9, the obtained Molecular weight for the P(CA₅-G), P(CA₈-G) and P(CA₁₂-G) is about 3000, 6000 and 8000, respectively. Table 1 shows the obtained molecular weight.

### Table-1 Showing the Molecular Weight for the P(CA₅-G), P(CA₈-G) and P(CA₁₂-G)

Sample

| | P(CA5-G) | P(CA8-G) | P(CA12-G) |
|---|---|---|---|
| Molecular Weight | 3000 | 6000 | 8000 |

These results indicate that the molecular weights of polyesters depend on the CA/G molar ratios and increase with an increase in the CA/G molar ratio. With increased CA/G molar ratio molecular weight of synthesized increase. The used citric acid/glycerol (CA/G) ratio for synthesizing of copolymers has a direct effect on their structural factors

The effect of polymerization time on molecular weight of P (CA12-G) was studied. In this regard, hyperbranched polyesters with a CA/G molar ratio of 12 and total reaction times of 4 and 8 h were synthesized and analyzed by using GPC. Table 2 shows the molecular weights of the polyester synthesized in different reaction times.

### Table-2 Showing the Molecular Weight for the P(CA₁₂-G) with Total Reaction Times of 4 h and 8 h

Reaction Time

| | 4 h | 8 h |
|---|---|---|
| Molecular weight | 8000 | 12000 |

As it can be seen in table 2, the molecular weights of synthesized hyperbranched polyesters depend on the total reaction times directly.

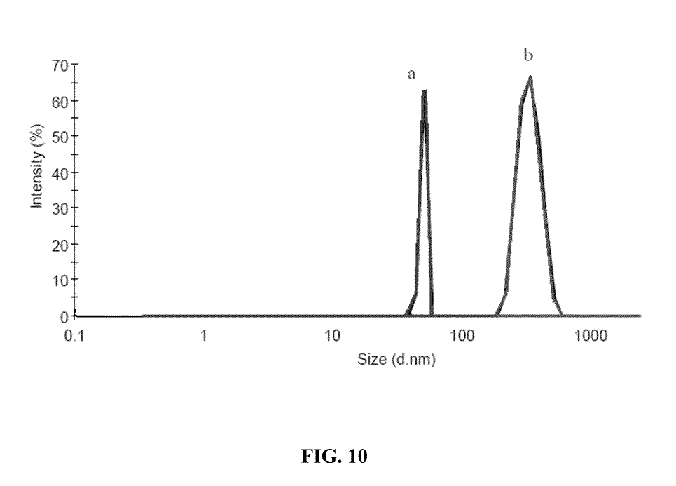

FIG. 10 shows a Dynamic light scattering (DLS) diagrams of the synthesized hyperbranched polymer with molar ratios of 8 and 12, where (a) shows the DLS diagram of the synthesized hyperbranched polymer with molar ratios of 8, P(CA₈-G) and where (b) shows the DLS diagram of the synthesized hyperbranched polymer with molar ratios of 12, P(CA₁₂-G), according to an embodiment herein. With respect to FIG. 10, it is clearly indicated that there is a direct relationship between size of hyperbranched polyesters and CA/G molar ratios.

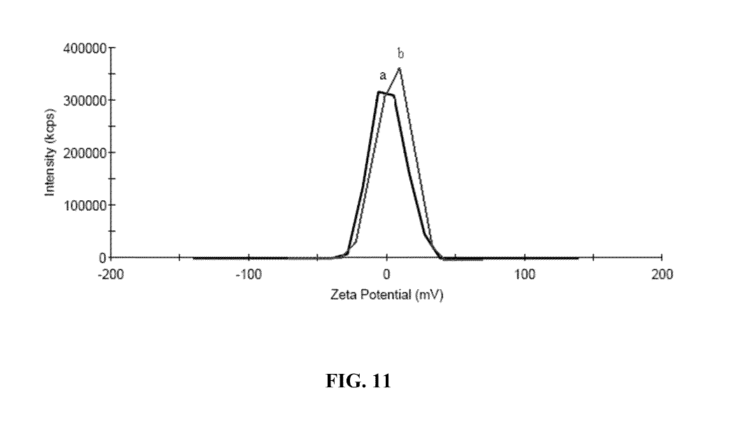

Zeta potential measurements were taken in water to obtain the information about surface charge of prepared polymers. FIG. 11 shows a graphical representation of the Zeta potential of synthesized hyperbranched polymer with molar ratios of 8 and 12, where (a) shows the zeta potential value for the synthesized hyperbranched polymer with a molar ratio of 8, P(CA₈-G) and where (b) shows the zeta potential value for the synthesized hyperbranched polymer with a molar ratio of 12, P(CA₁₂-G), according to an embodiment herein. With respect to FIG. 11, the diagrams show the variation in surface charge of polymers as a function of CA/G molar ratio. Another structural factor of synthesized copolymers which directly depends on the used CA/G molar ratio is the surface charge of copolymers. Clearly, an increase in the CA/G molar ratio leads to an increase in the negative surface charge.

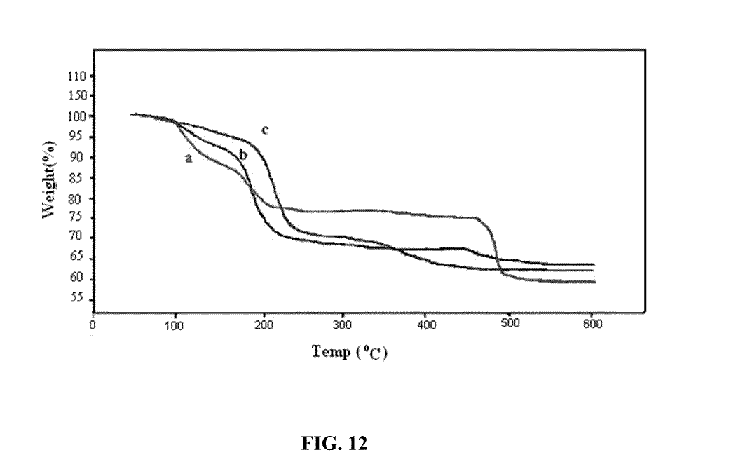

FIG. 12 shows a Thermo-gravimetric analyses (TGA) thermo grams of the synthesized hyperbranched polymers, where (a) shows a TGA thermogram for polymer with a molar ratio of 5, P(CA5-G), synthesized with a total reaction time of 4 h, where (b) shows a TGA thermogram for polymer with a molar ratio of 8, P(CA8-G), synthesized with total reaction time of 4 h and where (c) shows a TGA thermogram for polymer with a molar ratio of 5, P(CA5-G), synthesized with total reaction time of 6 h, according to an embodiment herein. With respect to FIG. 12, the weight loss of hyperbranched polyesters occurs in three stages at 95-110° C., 170-230° C. and 380-500° C. which are attributed to the evaporation of water, breaking of ester bonds and decomposition of sample, respectively. The second weight loss percentages in thermograms of (a), (b) and (c) are 10.5%, 19.5% and 20.5%, respectively. Therefore, TGA experiments confirm the NMR and GPC results. According to the experiments, the growth of synthesized hyper branched polyesters is affected by a broad range of parameters, such as CA/G molar ratio and reaction time.

Cisplatin was encapsulated into the interior void spaces of synthesized hyperbranched polyesters as mentioned before. The loading of cisplatin into the P(CA₈-G) and P(CA₁₂-G) was investigated by HPLC method. In this method, the standard curve of free cisplatin was obtained and used for calculating drug loading into the P(CA₈-G) and P(CA₁₂-G). Table 3 shows the results obtained by HPLC.

### Table 3 Showing the HPLC Results for the Encapsulation of CDDP into Void Spaces of P(CA₈-G) and P(CA₁₂-G)

| | First concentration | Free CDDP measured | Loading |
|---|---|---|---|
| Sample | of CDDP | by HPLC | capacity |
| P(CA₈-G) | 50 mg/ml | 6.01 mg/ml | 87.9% |
| P(CA₁₂-G) | 50 mg/ml | 6.4 mg/ml | 87.2% |

According to the HPLC results (table 3), the loading capacity of P(CA8-G) and P(CA12-G) is 87.9% and 87.2%, respectively.

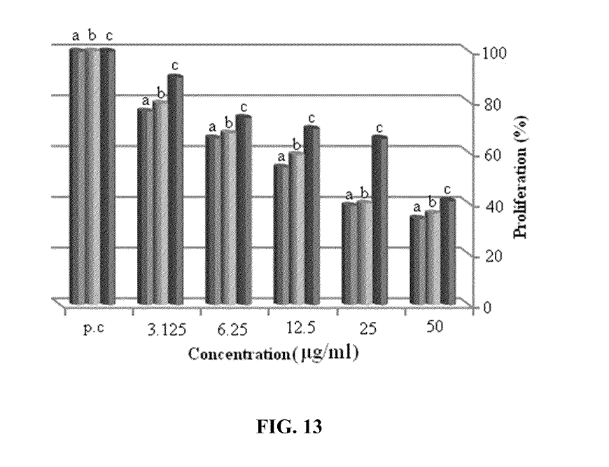

In vitro cytotoxicity of CDDP, P(CA₈-G)-CDDP and P(CA₁₂-G)-CDDP was evaluated by using a MTT assay. To measure cytotoxicity, tumor cells C26 were separately incubated in a plate with different concentrations of CDDP, P(CA₈-G)-CDDP and P(CA₁₂-G)-CDDP. The duration of incubation was 72 h. FIG. 13 shows a MTT (3-(4,5-dimethylthiazol-2-yl)-2,5-diphenyltetrazolium bromide) assay results for Cis-Diamminedichloroplatinum CDDP, P(CA₁₂-G)-CDDP and P(CA₈-G)-CDDP, wherein (a) shows graph for P(CA8-G)-CDDP, (b) shows graph for P(CA12-G)-CDDP and (c) shows graph for CDDP at different concentrations, according to an embodiment herein. With respect to FIG. 13, it is concluded that, loading of cisplatin into the P(CA₈-G)-CDDP and P(CA₁₂-G)-CDDP influences its cytotoxicity. P(CA₈-G)-CDDP and P(CA₁₂-G)-CDDP show higher toxicity as compared to free cisplatin. According to the MTT assay measurements, IC₅₀ doses of free cisplatin, P(CA₈-G)-CDDP and P(CA₁₂-G)-CDDP were calculated. IC₅₀ dose is the concentrations of active ingredients necessary to inhibit the cell growth by 50%. As shown in table 4, cisplatin loaded in polyesters has lower IC₅₀ value than the free cisplatin. In fact, a decrease in the IC₅₀ dose shows an increase in drug toxicity. Therefore, the acquired data show that the synthesized hyperbranched polyesters are used with good success as drug delivery systems.

### Table-4 Shows Obtained IC₅₀ Values for CDDP, P(CA₈-G)-CDDP and P(CA₁₂-G)-CDDP

| | Sample | | |
|---|---|---|---|
| | P(CA8-G)-CDDP | P(CA12-G)-CDDP | CDDP |
| IC₅₀ (μg/ml) | 15.01 | 16.8 | 37.9 |

The embodiments in their broader aspects and applications are not limited to the above embodiment and also directed to a large number of hyperbranched polyesters that may be formed from various monomers in different molar ratios of them.

Experimental Data

### Materials

Citric acid monohydrate, glycerol, tetrahydrofuran (THF), Cyclohexane and Cisplatin [cis-dichlorodiammineplatinum (II), CDDP] were purchased from Merck. 3-(4,5-Dimethylthiazol-2-yl)-2,5-diphenyltetrazolium bromide (MTT) powder and dialysis tubing of the molecular weight cut off of 1200 Da were purchased from Sigma Aldrich. The cell lines were obtained from the National Cell Bank of Iran (NCBI) Pasteur institute, Tehran, Iran.

### Instruments

¹H and ¹³C NMR spectra were recorded on a bruker DRX 400 (400 MHz) apparatus by using DMSO-d₆ and D₂O as was carried out in a thermal analyzer (model: DSC 60, shimadzu, Japan) under dynamic atmosphere of an inert gas (i.e. N2) at 30 ml/min (room temperature). The particle size was determined using Dynamic Light Scattering (DLS) (zetasizer ZS, Malvern Instruments). The molecular weight distributions were determined by size exclusion chromatography (SEC) using Pump 1000 using PL aquagel-OH mixed-H 8 μm column connected to a differential refractometer, RI with water as the mobile phase at 25° C. Pullulan standard samples were used for solvent calibration. FT-IR spectrum was recorded by a Nikolt 320 FT-IR. Thermo gravimetric analysis

### Example 1

### Production of Hyperbranched Polyesters Containing Citric Acid (CA) and Glycerol (G) Monomers with Different CA/G Molar Ratios, P(CA-G)

All hyperbranched polyesters used in the embodiments herein were synthesized by using citric acid monohydrate (CA) as AB3 monomer and glycerol (G) as A3 monomer at different CA/G molar ratios according to the melt polycondensation procedure. The used amounts of glycerol were 0.5 ml (6.6 mmol) and of citric acid monohydrate were 7 g (33 mmol), 11.09 g (52.8 mmol) and 16.64 g (79.2 mmol), corresponding to the CA/G molar ratios of 5, 8 and 12, respectively.

Citric acid monohydrate and glycerol were mixed in a polymerization ampule equipped with gas inlet, vacuum inlet and magnetic stirrer at 90° C. and heated to 110° C. for 20 min under constant stirring. The temperature of polymerization ampule was increased to 120° C. and mixture was stirred at this temperature for 30 min. Then the polymerization mixture was stirred at 130° C., 140° C., 145° C. and 150° C. for 40 min, 40 min, 50 min and 60 min, respectively under vacuum in order to remove the water formed during the reaction. The mixture was then kept at room temperature to cool down. Viscose compound was dissolved in tetrahydrofuran and filtered to obtain clear solution. The solution was then concentrated under the reduced pressure and product was precipitated in cyclohexane several times. Precipitated compound was dialyzed against THF for 4 h. Finally, THF was evaporated under the reduced pressure to obtain pure product as colorless and viscose compound. According to an embodiment herein, 5 Molar ratio is the best. However each compound prepared by each molar ratio has its own properties.

### Example 2

### Production of Hyperbranched Polyesters Containing Citric Acid (CA) and Glycerol (G) Monomers with Total Reaction Times of 6 h and 8 h

The total reaction time in example 1 was 4 h. Example 2 was synthesized in the same way as explained in Example 1 except that total reaction times were 6 h and 8 h instead of 4 h. According to an embodiment herein, 8 h is the best.

As mentioned before, the hyperbranched polyesters are promising candidates in order to use in variety of applications. For example to prove the efficacy of the hyperbranched polyesters as drug delivery systems, cisplatin (Cis-Diamminedichloroplatinum (CDDP) a platinum-based chemotherapy drug) was encapsulated into void spaces of the P(CA8-G) and P(CA12-G).

### Example 3

### Encapsulation of CDDP into Void Spaces of the P(CA-G), P(CA-G)-CDDP

0.1 g (1.67×10⁻² mmol) P(CA₈-G) was dissolved in 1 ml distilled water. Then 100 ml of cisplatin aqueous solution (50 μg/ml) was added drop-wise to the above solution. The solution stirred at 37° C. for 24 h in dark to obtain final product (P(CA₈-G)-CDDP) without any purification (97% yield).

P(CA₁₂-G)-CDDP was synthesized in the same manner as explained above. P(CA₈-G)-CDDP and P(CA₁₂-G)-CDDP were used for MTT assay measurements.

The proposed applications will be in extraction of heavy metal ions from liquids or water, detergents, food additives, plasticizers, composites and any products with a biodegradable property. The effects of molar ratio of citric acid/glycerol (CA/G) on the properties of obtained copolymer have been studied. For this reason, CA/G=5, 8 and 12 molar ratios were used to synthesize different copolymers. NMR, GPC, DLS and Zeta potential results showed that the properties of obtained copolymers, such as molecular weight, size and surface charge, was depended on the molar ratio of (CA/G). Based on these results, the molecular weight, size and surface charge of poly (citric acid-co-glycerol) copolymers increase with an increase in the (CA/G) molar ratio. This is explained by the fact that the average number of reacted citric acid monomers increases with increasing CA/G molar ratio. Applications of the obtained poly (citric acid-co-glycerol) copolymers should be influenced by changing these properties.

According to an exemplary embodiment of the present invention, hyperbranched polyesters based on citric acid as an AB3 monomer and glycerol as an A3 monomer at different citric acid/glycerol molar ratios were synthesized. It is clear that the use of citric acid and glycerol as building blocks has opened an opportunity for designing hyperbranched polyesters with high water solubility and biocompatibility. In fact, the poly (citric acid-co-glycerol) copolymers combine the advantages of both citric acid and highly branched polymers. Therefore, it is suggested that these materials are used for a wide variety of applications. Some of proposed applications in which poly (citric acid-co-glycerol) copolymers are used are in medicine, in blends and as a metal ion extractant.

In medicine, HBs are applied as drug carrier molecules. In drug delivery systems based on HBs, a drug molecule is either non-covalently transported or covalently conjugated to their surface functional groups. In chemical conjugation, drug molecules are attached to the surface functional groups of HBs via direct conjugation or via a linker molecule, if the drugs do not carry the desired functional group for direct conjugation. On the other hand, several drug molecules and targeting groups are conjugated to the surface groups of HBs because HBs possess controlled multi-valency.

In blends, macromolecules with a compact architecture are of great interest as additives or as building blocks in novel polymeric materials. Among them, hyperbranched polymers represent an important part of the family of dendritic and multi-branched polymers. HBs possess very high solubilities and much lower solution viscosities compared to those of linear polymers, which result from large number of peripheral terminal functional groups available per macromolecule and their packed structure. In general, HBs blended with different linear polymers are used to improve physical and chemical properties of linear polymers such as thermal stability, melt viscosity and solubility. The properties of HBs in blends are indeed strongly determined by the nature of their terminal groups.

As metal ion extractant, for polymer supported ultrafiltration (PSUF) which is emerging as a promising process for the treatment of water contaminated with toxic metal ions, hyperbranched polymers may be good candidates. This is because the efficiency of PSUF is dependent on binding of pollutant to the polymer and sorption of the polymer onto ultrafiltration membrane. Consequently, the availability of polymers with large metal binding capacities and weak sorption tendencies on membrane is critical in the development of cost effective PSUF processes. Thus water-soluble HBs with chelating functional groups and surface groups having weak binding affinity toward ultrafiltration membranes are expected to be good candidates for PSUF and may open unprecedented opportunities in this context.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A hyper branched polyester comprising:
an acidic moiety; and
an alcoholic moiety.

2. The polyester according to claim 1, wherein acidic moiety comprises at least three acidic functional groups.

3. The polyester according to claim 1, wherein the acidic moiety comprises one or more types of acidic compounds.

4. The polyester according to claim 1, wherein the acidic moiety comprises one type of acidic compound.

5. The polyester according to claim 1, wherein the acidic moiety is citric acid monohydrate.

6. The polyester according to claim 1, wherein the alcoholic moiety comprises at least three hydroxyl functional groups.

7. The polyester according to claim 1, wherein the alcoholic moiety comprises one or more types of alcoholic compounds.

8. The polyester according to claim 1, wherein the alcoholic moiety comprises one type of alcoholic compound.

9. The polyester according to claim 1, wherein the alcoholic moiety is glycerol.

10. The polyester according to claim 1, wherein the acidic moiety and the alcoholic moiety are randomly arranged.

11. A method of synthesizing a poly(citric acid-co-glycerol) polymer, comprising the steps of:
mixing a predetermined amount of citric acid monohydrate (CA) and a predetermined amount of glycerol (G) in a polymerization ampule to prepare a first solution, wherein the citric acid and glycerol are mixed at a temperature of 90° C.;
heating the prepared first solution at a temperature of 110° C. for 20 min by constantly stirring the first solution;
increasing a temperature of the first solution in the polymerization ampule further to 120° C. and stirring the first solution simultaneously for next 30 min;
stirring the first solution at a plurality of temperature levels for a plurality of time intervals successively; wherein the first solution is stirred at 130° C. for 40 min; wherein the first solution is stirred at 140° C. for 40 min; wherein the first solution is stirred at 145° C. for 50 min and wherein the first solution is stirred at 150° C. for 60 min, and wherein the first solution is heated and stirred successively at the plurality of temperature levels under vacuum to remove a water;
keeping the first solution at a room temperature to cool down to form a viscose compound;
dissolving the viscose compound in tetrahydrofuran (THF) to form a second solution and wherein the second solution is a viscous solution;
filtering the second solution to obtain a third solution and wherein the third solution is a clear solution;
concentrating the third solution under a reduced pressure to obtain a second compound;
precipitating the second compound in a cyclohexane solvent;
dialysing the precipitated second compound against a THF solvent to obtain a fourth solution;
evaporating the fourth solution under a reduced pressure to obtain a pure product of hyper branched polyester and wherein the hyper branched polyester is obtained in different molar ratios to obtain hyper branched polyester with different sizes and with different surface charges.

12. The method according to claim 11, wherein the citric acid monohydrate and glycerol are mixed in a plurality of molar ratios of citric acid monohydrate/glycerol (CA/G) and wherein the plurality of molar ratios of citric acid monohydrate/glycerol (CA/G) includes 5, 8 and 12.

13. The method according to claim 11, wherein the citric acid monohydrate and glycerol are mixed in a molar ratio of 5.

14. The method according to claim 11, wherein the predetermined amount of citric acid monohydrate is 7 g (33 mmol) or 11.09 g (52.8 mmol) or 16.64 g (79.2 mmol).

15. The method according to claim 11, wherein the predetermined amount of glycerol is 0.5 ml (6.6 mmol).

16. The method according to claim 11, wherein the precipitated compound is dialyzed against THF solvent for 4 h, 6 h and 8 h, and wherein the precipitated compound is against THF solvent dialyzed for 8 h.

17. A method of encapsulating a drug into a hyperbranched polyester comprising the steps of:
dissolving a hyper branched polyester in a distilled water, wherein 0.1 gm (1.67×10⁻² mmol) of hyperbranched polyester is dissolved in 1 ml distilled water;
making a drug solution with a preset quantity at a preset concentration and wherein the preset quantity is 100 ml and wherein the preset concentration is 50 μg/ml;
adding the drug solution to the dissolved hyper branched polyester solution to obtain a mixture solution and wherein the drug solution is added the dissolved hyper branched polyester solution in drop-wise;
stirring the mixture solution at 37° C. for 24 h in a dark condition to obtain an encapsulated drug;
wherein drug molecules are encapsulated into a void space of the hyperbranched polyester in the encapsulated drug.

18. The method according to claim 17, wherein the hyperbranched polyester is obtained by mixing a preset quantity of citric acid and a preset quantity of glycerol in a molar ratio of 5, 8 and 12, and wherein the preset quantity of citric acid is 7 g (33 mmol) or 11.09 g (52.8 mmol) or 16.64 g (79.2 mmol) and wherein the preset quantity of glycerol is 0.5 ml (6.6 mmol).

19. The method according to claim 17, wherein the drug is cisplatin.
